(19) United States
(12) Patent Application Publication
(10) Pub. No.: US 2018/0344646 A1
(43) Pub. Date: Dec. 6, 2018

(54) AMORPHOUS DISPERSION GRANULES AND ORAL DOSAGE FORMS

(71) Applicant: PATHEON DEVELOPMENT SERVICES INC., Bend, OR (US)

(72) Inventors: Sanjay Konagurthu, Bend, OR (US); Thomas REYNOLDS, Bend, OR (US); Marshall CREW, Bend, OR (US)

(73) Assignee: PATHEON DEVELOPMENT SERVICES INC., Bend, OR (US)

(21) Appl. No.: 15/778,322

(22) PCT Filed: Nov. 14, 2016
(86) PCT No.: PCT/US2016/061840

§ 371 Date: May 23, 2018

Related U.S. Application Data
(60) Provisional application No. 62/259,871, filed on Nov. 25, 2015.

(51) Int. Cl. A61K 9/16; A61K 31/4422; A61K 31/405

(52) U.S. Cl. CPC A61K 9/1652; A61K 31/405; A61K 31/4422; A61K 9/1623

(57) ABSTRACT

Amorphous dispersion granules comprising an amorphous solid solution of an active pharmaceutical ingredient and a dispersing polymer, and deposited thereon a substrate comprising at least one first tableting agent, as well as oral dosage forms comprising the amorphous dispersion granules of the invention, including pharmaceutically acceptable tablets comprising the amorphous dispersion granules of the invention, and methods of preparing amorphous dispersion granules and pharmaceutically acceptable tablets manufactured therefrom.

Pharmaceuticals are often formulated from crystalline compounds because they typically provide high levels of purity and are resistant to physical and chemical instabilities under ambient conditions. The use of crystalline compounds in formulations has become increasingly challenging because many chemical entities are trending towards having higher molecular weight, greater lipophilicity and lower aqueous solubility, all of which negatively affect oral bioavailability. Unlike a crystalline solid, which has an orderly array of unit cells in three dimensions, amorphous solids lack long-range order because molecular packing is more random. As a result, amorphous compounds tend to have different properties than their crystalline counterparts. For example, amorphous compounds are more soluble than crystalline forms of the same compound (see, e.g., Hancock and Parks, Pharmaceutical Res. 17, 397 (2000)), which is why, in pharmaceutical formulations whose crystalline forms are poorly soluble, amorphous compounds may present attractive formulation options. As such, amorphous active pharmaceutical ingredients (APIs) are increasingly used to improve certain physical and chemical properties of drugs, such as, for example, dissolution and bioavailability.

It is useful to understand the intrinsic physicochemical and biopharmaceutical properties of the APIs prior to or at the onset of drug development. To that end, the biopharmaceutical classification system (BCS) has been routinely utilized to assess oral absorption and guide formulation development. Formulating oral dosage forms of APIs in BCS class II and class IV is especially challenging. Class II encompasses compounds of high permeability and low solubility whereas class IV encompasses compounds of low permeability and low solubility. With such compounds, pharmaceutical formulators often turn to the amorphous form of APIs to increase the solubility. However, because crystalline forms are more thermodynamically stable than amorphous forms, there is a driving force toward crystallization of the amorphous state for any given compound or mixtures of compounds. Therefore, when preparing amorphous drug products in the form of tablets or other finished dosage form, it is often desirable to prepare an intermediate composition, prior to making a finished dosage form, of the active pharmaceutical ingredient with an excipient that stabilizes the amorphous state against crystallization. This kind of composition may be referred to as an amorphous dispersion, where an amorphous API being dispersed within an excipient, often a polymer. A common method of making such a dispersion is through spray drying. As used herein, such a spray-dried dispersion made according to standard methods in the art is referred to as a spray-dried intermediate (“SDI”).

SDIs may be prepared by dissolving one or more APIs in one or more polymers in solvents followed by a spray-drying process. The physical and mechanical properties of SDIs are not suitable, however, to directly compress into tablets. For example, there is often substantial particle-size inhomogeneity in SDIs and they often lack suitable flow properties and compressibility for direct compression into tablets.

Multiple unit operations are used to process SDIs into compressible materials suitable for tableting. Such unit operations typically involve initially blending the SDI with all intragranular components, other than a lubricant such as magnesium stearate, which is added later. Another unit operation involves delumping the resulting mixture by passing through a sieve. To maintain content uniformity, further blending may be required. A lubricant may then be added. Such lubricants are critical to the success of tableting SDIs. After the lubricant is added yet another blending step is often needed. Finally, after multiple unit operations, tablets may be prepared by compressing the final blend. It is not uncommon, therefore, to have multiple drying, blending, dry granulation, milling, and lubrication operations when formulating SDIs into pharmaceutically acceptable tablets. Such operations are time consuming and costly.

### SUMMARY OF INVENTION

In one aspect of the invention, amorphous dispersion granules comprising an amorphous solid solution of an active pharmaceutical ingredient and a dispersing polymer, and deposited thereon a substrate comprising at least one first tableting agent, are provided.

In another aspect of the invention, oral dosage forms comprising the amorphous dispersion granules of the invention are provided.

In yet another aspect of the invention, pharmaceutically acceptable tablets comprising the amorphous dispersion granules of the invention are provided.

In a further aspect of the invention, methods for preparing amorphous dispersion granules comprising the steps of dissolving an active pharmaceutical ingredient and a dispersing polymer in a solvent to form a granulation binder; and spraying the granulation binder onto a fluidized substrate comprising at least one tableting agent to form an amorphous dispersion granule are provided.

In a further aspect of the invention, methods of preparing pharmaceutically acceptable tablets comprising directly compressing a composition comprising amorphous dispersion granules of the invention are provided.

### DETAILED DESCRIPTION

The amorphous dispersion granule (also referred to as “ADG”) of the invention possesses a unique morphology differing from the morphology of the conventional amorphous dispersion granules. As indicated above for SDIs, conventional ADGs and first tableting agents are physically mixed, whereas the amorphous solid solution is deposited onto the first tableting agent in the ADGs of the invention. This specific morphology generally provides the ADGs of the invention to have better free flowing properties and an improved compressibility and material properties compared to conventional SDIs. The mechanical properties of the ADGs and their corresponding tablets can be tuned as desired, e.g. by adjusting the ratio of the active pharmaceutical ingredient and the dispersing polymer. In addition, the ADGs of the invention can be compressed into pharmaceutically acceptable tablets much easier, thereby requiring less compression forces while maintaining good or even better distribution of the active pharmaceutical ingredient. Moreover, the tableting process becomes simpler as it requires fewer processing steps, and hence the production of tablets from ADGs is generally commercially and economically more attractive.

Characterization of the ADGs may be done by any suitable analytical technique known in the art, in particular techniques such as x-ray powder diffraction, modulated differential scanning calorimetry (“mDSC”), infrared spectroscopy, raman spectroscopy, solid-state nuclear magnetic resonance spectroscopy, various microscopic techniques such as scanning electron microscopy (“SEM”) optionally in combination with elemental analysis such as EDX, and density measurements such as tap density and bulk density can be suitably used. SEM micrographs are useful for evaluating particle morphology such as size and uniformity of structure. When using mDSC, one tends to look for a single glass transition temperature which indicates that the material is a dispersion as opposed to phase-separated mixture.

The amorphous dispersion granule of the invention has a particle size distribution suitable for tableting purposes. The ADG generally has a d90 value of at least 1 □m, preferably at least 10 □m, more preferably at least 20 □m, even more preferably at least 50 □m, and most preferably at least 100 □m, and generally at most 1000 □m, preferably at most 500 □m, more preferably at most 400 □m, even more preferably at most 300 □m, and most preferably at most 200 □m. The “d90 value” has its common meaning and generally refers to a particle size distribution wherein 90% of the ADG particles have a particle size below the given d90 value. Such d90 values can be determined using standard techniques.

The amorphous solid solution comprises an active pharmaceutical ingredient (API) and a dispersing polymer. The API in its pure form is generally crystalline and poorly soluble. In the solid solution the API is dissolved in the dispersing polymer and/or stabilized by the dispersing polymer. Generally, the API is in an amorphous state, i.e. no detectable crystalline API is present in the solid solution. The amorphous state of the API can be obtained through a molecular dispersion at a concentration below saturation or at a supersaturation concentration, or though discrete amorphous particles (lacking long range order), or a combination thereof. Determination of the amorphous state can be performed using X-ray diffraction; the absence of sharp peaks in the X-ray diffractogram is indicative of the API being in an amorphous state. Additionally, the solid solution has one glass transition temperature when measured using DSC. Preferably, the amorphous solid solution exhibits one glass transition temperature and comprises the API in an amorphous state. The solid dispersions disclosed in the prior art describe dispersions comprising a plurality of different phases and/or the presence of API crystallites. Examples of such prior art includes US 2010/0015225, WO 2005/070397 and WO 2008/065674. The dispersion granulates comprising prior art dispersions will generally have an API solubility profile different from the ADG, and additionally an inferior compressibility, compactability and/or tabletability. Additionally, the presence of such phases and/or crystallites may cause difficulties in obtaining uniform ADGs and corresponding tablets, and/or may render manufacturing of such tablets more difficult.

The ADG of the invention generally have superior compressibility, compactibility and tabletability compared to an SDI physically mixed with the substrate and dry granulated. The SDI together with the substrate will comprise the same ingredients in similar amounts as the ADG of the invention. Preferably, the compactibility of the ADG of the invention is at least 105% of the compactibility of the SDI, more preferably at least 110%, even more preferably at least 120% and most preferably at least 130%. The improved tableting properties allows for a lower compression force to obtain a comparable tensile strength and/or hardness of the resulting tablet.

The weight ratio of API and dispersing polymer is chosen such that the API will be dispersed in the dispersing polymer to form the amorphous solid solution. The skilled person will understand that the maximum ratio will depend on the API and dispersing polymer used. Generally, the weight ratio of API and dispersing polymer is at least 1:20, preferably at least 1:10, more preferably at least 1:5, even more preferably at least 1:3 and most preferably at least 1:2, and generally at most 20:1, preferably at most 10:1, more preferably at most 5:1, even more preferably at most 3:1 and most preferably at most 2:1.

The active pharmaceutical ingredient can be any API known in the art which can be suitably used in ADGs. The API can have any solubility as long as the API is able to form an amorphous solid solution with the dispersing polymer. In one embodiment, such APIs are BCS Class II compounds and BCS Class IV compounds. In one embodiment of the invention, the API has a solubility in water at 25° C. and a pH of 7.4 of at most 1 mg/ml, preferably at most 500 □g/ml, more preferably at most 100 □g/ml, even more preferably at most 50 □g/ml, even more preferably at most 10 □g/ml, even more preferably at most 5 □g/ml, even more preferably at most 1 □g/ml, and most preferably at most 0.5 □g/ml. Examples of suitable APIs include alprazolam, amiodarone, amlodipine, amprenavir, aprepitant, aripiprazole, astemizole, atenolol, auranofin, azathioprine, azelastine, beclomethasone, belsomra, bexarotene, biperiden, boceprevir, budesonide, buprenorphine, butalbital, calcitriol, carbamezapine, carbidopa, carvedilol, cefotaxime, cephalexin, chlorpromazine, cholestyramine, ciprofloxacin, cisapride, cisplatin, clarithromycin, clofazimine, cefuroxime, clemastine fumarate, clonazepam, clozapine, cyclandelate, cyclosporin, danazol, diazepam, diclofenac, diclofenac sodium, digoxin, dipyridamole, divalproex, dobutamine, doxazosin, doxercalciferol, dronabinol, dutasteride, efavirenz, emalapril, estradiol, etodolac, etoposide, etravirine, everolimus, famotidine, felodipine, fenofibrate, fenoprofen, fentanyl citrate, fexofenadine, finasteride, fluconazole, flunosolide, fluphenazine, flurbiprofen, fluvoxamine, furosemide, glipizide, glyburide, glibendamine, griseofulvin, hydrocortisone, ibuprofen, indomethacin, isosorbide dinitrate, isotretinoin, isradipine, itaconazole, ivacaftor, ketoconazole, ketoprofen, lamotrigine, lansoprazole, ledipasivir, loperamide, lopinavir, loratidine, lorazepam, lovastatin, maropitant, medroxyprogesterone, mefenamic acid, megestrol acetate, mesalamine, methylprednisolone, miconazole, midazolam, mitomycin, mometasone, nabilone, nabumetone, naproxen, nelfinavir mesylate, nicergoline, nifedipine, nimodipine, nintedanib, nitrofurantoin, norfloxacin, nufenamic acid, olaparib, oleanolic acid, omeprazole, paclitaxel, paliperidone, perphenazine, phenytoin, piroxicam, posaconazole, quinapril, quinapril hydrochloride, ramipril, retinol palmitate, risperidone, ritonavir, paracetamol, pericalcitol, praziquantel, saquinavir, sertraline, simeprevir, simvastatin, sirolimus, spironolactone, sulfasolizine, tacrolimus, telaprevir, terbinafine, terfernadine, testosterone, testosterone undecanoate, tipranavir, tolterodine tartrate, tretinoin, triamcinolone, valproic acid, vemurafenib, verapamil, voraconazole, zafirlukast, ziprasidone, and zolpidem.

The amorphous dispersion granule comprises the API in an amount of at least 0.1 weight percent (wt %), preferably at least 1 wt %, more preferably at least 5 wt % and most preferably at least 10 wt %, based on the total weight of the ADG, and generally at most 75 wt %, preferably at most 60 wt %, more preferably at most 50 wt % and most preferably at most 40 wt %, based on the total weight of the ADG. The total weight (or weight percent) of all the ingredients present in the ADG add up to 100 wt %.

The dispersing polymer can be any polymer known in the art that can be suitably used to dissolve API and form a solid solution. Examples of suitable dispersing polymers include hydroxypropyl methyl cellulose succinate, polyvinyl acetate phthalate, Soluplus (polyvinyl caprolactam-polyvinyl acetate-polyethylene glycol graft copolymer (PCL-PVAc-PEG)), Vinylpyrrolidone-vinyl acetate copolymer (Copovidone), polyvinyl pyrrolidone (povidone), cellulose acetate succinate, methyl cellulose acetate succinate, ethyl cellulose acetate succinate, hydroxypropyl cellulose acetate succinate, hydroxypropyl methyl cellulose acetate succinate (HPMCAS), hydroxypropyl cellulose acetate phthalate succinate, cellulose propionate succinate, hydroxypropyl cellulose butyrate succinate, hydroxypropyl methyl cellulose phthalate (HPMCP), cellulose acetate phthalate, methyl cellulose acetate phthalate, ethyl cellulose acetate phthalate, hydroxypropyl cellulose acetate phthalate, hydroxypropyl methyl cellulose acetate phthalate, cellulose propionate phthalate, hydroxypropyl cellulose butyrate phthalate, cellulose acetate trimellitate, methyl cellulose acetate trimellitate, ethyl cellulose acetate trimellitate, hydroxypropyl cellulose acetate trimellitate, hydroxypropyl methyl cellulose acetate trimellitate, hydroxypropyl cellulose acetate trimellitate succinate, cellulose propionate trimellitate, cellulose butyrate trimellitate, cellulose acetate terephthalate, cellulose acetate isophthalate, cellulose acetate pyridinedicarboxylate, salicylic acid cellulose acetate, hydroxypropyl salicylic acid cellulose acetate, ethylbenzoic acid cellulose acetate, hydroxypropyl ethylbenzoic acid cellulose acetate, ethyl phthalic acid cellulose acetate, ethyl nicotinic acid cellulose acetate, ethyl picolinic acid cellulose acetate, carboxy methyl cellulose, carboxy ethyl cellulose, ethyl carboxy methyl cellulose, hydroxypropyl methyl cellulose acetate, hydroxypropyl methyl cellulose (HPMC), hydroxypropyl cellulose, methyl cellulose, hydroxyethyl methyl cellulose, hydroxyethyl cellulose acetate, and hydroxyethyl ethyl cellulose, copolymers of methacrylates, copolymers of acrylates, poly(methacylic acid-co-methyl methacrylate), alone or in combination. Preferably, the dispersing polymer is selected from polyvinyl pyrrolidone (PVP), polyvinyl alcohol (PVA), polyvinyl pyrrolidone-polyvinyl acetate copolymer (copovidone, PVP-VA), polyvinyl acetate (PVAP), hydroxypropyl methylcellulose (HPMC), hydroxypropyl cellulose (HPC), carboxymethyl ethyl cellulose (CMEC), cellulose acetate phthalate (CAP), cellulose acetate trimellitate (CAT), hydroxypropyl methyl cellulose phthalate (HPMCP), hydroxypropyl methyl cellulose acetate succinate (HPMCAS), acrylate and methacrylate (co)polymers, polyethylene glycol (PEG), polyethylene oxide (PEO), and polyethylene-polypropylene glycol copolymer.

The amorphous dispersion granule comprises the dispersing polymer in an amount of at least 0.1 weight percent (wt %), preferably at least 1 wt %, more preferably at least 5 wt %, even more preferably at least 10 wt %, and most preferably at least 20 wt %, based on the total weight of the ADG, and generally at most 99 wt %, preferably at most 95 wt %, more preferably at most 90 wt % and most preferably at most 80 wt %, based on the total weight of the ADG.

The amorphous solid solution of the invention may further comprise first excipients. Examples of suitable excipients include tableting agents such as ductile components, brittle components, and disintegrants; surfactants, glidants, lubricants, wetting agents, pH adjusting agents, antioxidants, precipitation inhibitors, flavoring or food additives, coloring agents, stabilizers, binders, odor controlling agents, and preservatives. A combination of two or more first excipients can be comprised in the amorphous solid solution. In a preferred embodiment, the solid solution further comprises a second tableting agent. In one aspect, the second tableting agent is a disintegrant.

The amorphous dispersion granule comprises the first excipient, preferably the second tableting agent and more preferably the disintegrant, in an amount of at least 0.1 weight percent (wt %), preferably at least 0.5 wt %, more preferably at least 1 wt % and most preferably at least 2 wt %, based on the total weight of the ADG, and generally at most 20 wt %, preferably at most 15 wt %, more preferably at most 10 wt % and most preferably at most 5 wt %, based on the total weight of the ADG.

The ADG further comprises the amorphous solid solution deposited on the substrate. The substrate comprises a first tableting agent. The substrate may comprise two or more first tableting agents. The further embodiments given below for the first tableting agent apply mutatis mutandis for the second tableting agent. The first tableting agent may be the same or different from the second tableting agent.

The amorphous dispersion granule comprises the first tableting agent in an amount of at least 0.1 weight percent (wt %), preferably at least 0.5 wt %, more preferably at least 1 wt % and most preferably at least 2 wt %, based on the total weight of the ADG, and generally at most 60 wt %, preferably at most 50 wt %, more preferably at most 45 wt % and most preferably at most 40 wt %, based on the total weight of the ADG.

The first tableting agent may be a brittle component, a ductile component or a disintegrant. In one embodiment the substrate comprises a ductile and brittle component. This is particularly preferred embodiment when the second tableting agent is a disintegrant. In another embodiment, the substrate comprises a ductile component, a brittle component and a disintegrant. The term “ductile component” is to be given its ordinary and customary meaning to a person of ordinary skill in the art (and not limited to a special or customized meaning), and refers to a pharmaceutically acceptable excipient that is capable of deforming under tensile stress. Examples of ductile components as used herein include, but are not limited to, one or more of microcrystalline cellulose and starch. Other examples of ductile components can be found in industry standard textbooks and handbooks such as “Handbook of Pharmaceutical Excipients, Edited by Raymond C. Rowe, 7th ed, 2012.

The term “brittle component” is to be given its ordinary and customary meaning to a person of ordinary skill in the art (and not limited to a special or customized meaning), and refers to a pharmaceutically acceptable excipient that breaks without significant deformation. Brittleness is a property whereby materials, such as ceramics and glasses, fracture without substantial deformation upon absorption of energy. Examples of brittle components as used herein include, but are not limited to, one or more of mannitol, lactose, calcium phosphate, Starch 1500, silicas and silicates. Other examples of such brittle components can be found in industry standard textbooks and handbooks such as “Handbook of Pharmaceutical Excipients, Edited by Raymond C. Rowe, 7th ed, 2012.

The term “disintegrant” is to be given its ordinary and customary meaning to a person of ordinary skill in the art (and not limited to a special or customized meaning), and refers to a pharmaceutically acceptable excipient that expands and dissolves when wet. When part of a tablet, this action causes the tablet to break apart after ingestion which assists in the release of an active ingredient. Examples of disintegrants as used herein include, but are not limited to, one or more of croscarmellose sodium such as AcDiSol, cross-linked carboxymethyl cellulose, guar gum, cross-linked polyvinyl pyrrolidone (PVP) such as PVP-XL, hydroxypropyl cellulose such as L-HPC, soy polysaccharides, clays such as hydrotalcite and bentonite, and starches like sodium starch glycolate. Other examples of such disintegrants can be found in industry standard textbooks and handbooks such as “Handbook of Pharmaceutical Excipients, Edited by Raymond C. Rowe, 7th ed, 2012.

The substrate of the invention may further comprise second excipients commonly used in tableting processes. Examples of suitable excipients include surfactants, glidants, lubricants, wetting agents, pH adjusting agents, antioxidants, precipitation inhibitors, flavoring or food additives, coloring agents, stabilizers, binders, odor controlling agents, and preservatives.

The amorphous dispersion granule comprises the second excipient in an amount of at least 0.1 weight percent (wt %), preferably at least 0.5 wt %, more preferably at least 1 wt % and most preferably at least 2 wt %, based on the total weight of the ADG, and generally at most 20 wt %, preferably at most 15 wt %, more preferably at most 10 wt % and most preferably at most 5 wt %, based on the total weight of the ADG.

The invention further pertains to oral dosage forms comprising the ADG of the invention. The oral dosage form can be any one selected from tablets, capsules, caplets, powders, pellets, granules and suspensions.

The invention further pertains to pharmaceutically acceptable tablets comprising the ADG of the invention.

The invention further pertains to a method of preparing amorphous dispersion granules comprising the steps of dissolving an active pharmaceutical ingredient and a dispersing polymer in a solvent to form a granulation binder; and spraying the granulation binder onto a fluidized substrate comprising at least one tableting agent to form an amorphous dispersion granule.

The granulation binder comprises an API, a dispersing polymer and a solvent. The API and the dispersing polymer are as defined above. The solvent may be any solvent known in the art and suitable for use in the granulation binder of the invention. In one aspect, the granulation binder may comprise two or more solvents. The solvent typically is capable of dissolving both the API and the dispersing polymer. Examples of suitable solvents include acetone, methanol, ethanol, isopropyl alcohol, tetrahydrofuran (THF), methylene chloride, water, methyl ethyl ketone, acetonitrile, ethyl acetate, dimethyl sulfoxide, N-methyl-2-pyrrolidone, dimethyl formamide and other ICH defined solvents for use in the production of medicinal products.

The amount of solvent typically used in the granulation binder of the invention is sufficient to allow for spraying droplets of the granulation binder onto the substrate to enable agglomeration of the amorphous solid solution and the first tableting agent. Using too much solvent will lead to larger and non-uniform agglomerate formation. The right amount of solvent will depend on the API and the dispersing polymer. The granulation binder generally comprises the solvent in an amount of at least 1 weight percent (wt %), preferably at least 5 wt %, more preferably at least 10 wt % and most preferably at least 20 wt %, based on the total weight of the granulate binder, and generally at most 60 wt %, preferably at most 50 wt %, more preferably at most 40 wt % and most preferably at most 30 wt %, based on the total weight of the granulate binder.

The granulation binder comprises the API in an amount of at least 0.1 weight percent (wt %), preferably at least 0.5 wt %, more preferably at least 1 wt % and most preferably at least 2 wt %, based on the total weight of the granulation binder, and generally at most 99 wt %, preferably at most 90 wt %, more preferably at most 80 wt % and most preferably at most 70 wt %, based on the total weight of the granulate binder.

The granulation binder comprises the dispersing polymer in an amount of at least 0.1 weight percent (wt %), preferably at least 0.5 wt %, more preferably at least 1 wt % and most preferably at least 2 wt %, based on the total weight of the granulation binder, and generally at most 99 wt %, preferably at most 90 wt %, more preferably at most 80 wt % and most preferably at most 70 wt %, based on the total weight of the granulate binder.

The granulation binder may further comprise first excipients as indicated above for ADGs. The granulation binder comprises the first excipient, preferably the second tableting agent, more preferably the disintegrant, in an amount of at least 0.1 weight percent (wt %), preferably at least 0.5 wt %, more preferably at least 1 wt % and most preferably at least 2 wt %, based on the total weight of the granulation binder, and generally at most 20 wt %, preferably at most 15 wt %, more preferably at most 10 wt % and most preferably at most 5 wt %, based on the total weight of the granulate binder. The total weight (or weight percentage) of all ingredients in the granulation binder add up to 100 wt %.

ADGs may be prepared by spraying the granulation binder onto a fluidized bed substrate (or “substrate”). The substrate is as described above. The spraying of the granulation binder can be performed with any suitable spraying method known in the art. Typically the spraying is performed using an atomizing nozzle such as a two-fluid nozzle. The substrate is maintained in a fluidized state in a fluidized bed while the granulation binder is sprayed onto the substrate particles. In one aspect, a plurality of substrates is used in the process of the invention. The solvent in the granulation binder renders the API/dispersing polymer to become sticky or tacky resulting in agglomeration to the substrate(s). Simultaneously, the solvent is evaporated while maintaining the resulting ADG particles in the fluidized state. The fluidization of the substrate can be performed in any apparatus known in the art used to fluidize particles and form a fluidized bed. An example of such an apparatus is a fluidized bed dryer such as apparatus creating a circulating fluidized bed, a vibratory fluidized bed or an annular fluidized bed. The granulation binder may be sprayed onto the fluidized substrate in a fluid-bed dryer wherein the spray process is in a top-spray mode, bottom-spray mode, or a tangential spray mode. In the method of the invention the solvent is at least partially or completely evaporated to form the amorphous dispersion granules of the invention.

The substrate components may be fluidized by using parameters well known to those of ordinary skill in the art. Indeed, commercial fluid bed dryers are readily available for purchase. Fluidization is typically achieved by adjusting temperature and the flow of a gas such as air, clean dry air, nitrogen, humidified air, reactive gases, or a combination thereof until the substrate in the fluid bed dryer or other apparatus exposed to said temperature and gas behaves as a fluid rather than a solid. In particular, such behavior may manifest itself in that the solid flows like a fluid. Such “free flow” is indicative of fluidized substrate. In many embodiments of the invention, the mechanical properties of the ADGs may be adjusted by changing the API and/or the polymer and/or substrate components and ratios. Such mechanical properties include flowability, compressibility, compactibility, and tabletability. Other features that may be adjusted based on the ratio of components employed include the ability to tune dissolution and/or absorption properties.

The invention further pertains to methods of preparing pharmaceutically acceptable tablets comprising compressing a composition comprising amorphous dispersion granules of the invention. The compressing can be performed using any conventional tableting apparatus under conditions known by the skilled person. As indicated the pressure can be reduced when using the ADGs of the invention.

As used herein, “oral dosage form” refers to a formulation that is prepared for administration to a subject through the oral route of administration. Examples of known oral dosage forms, include without limitation, tablets, capsules, caplets, powders, pellets, granules, solutions, suspensions, solution pre-concentrates, and emulsions and emulsion pre-concentrates, In some aspects, powders, pellets, granules and tablets may be coated with a suitable polymer or a conventional coating material to achieve, for example, greater stability in the gastrointestinal tract, or to achieve the desired rate of release. Capsules containing a powder, pellets or granules may be further coated. Tablets may be scored to facilitate division of dosing. The oral dosage forms described herein are useful in the delivery of APIs for the treatment of disease or other conditions.

The term “pharmaceutically acceptable excipient” or “excipient” is to be given its ordinary and customary meaning to a person of ordinary skill in the art and not limited to a special or customized meaning), and refers to an excipient which may be used in the commercial manufacture of pharmaceutically acceptable tablets.

The term “pharmaceutically acceptable tablet” is to be given its ordinary and customary meaning to a person of ordinary skill in the art and not limited to a special or customized meaning), and refers to a tablet that has been prepared so that it would be suitable for manufacture for ultimate patient use. Such tablets would be suitably hard for typical commercial use, for example.

The term “precipitation inhibitor” is to be given its ordinary and customary meaning to a person of ordinary skill in the art (and not limited to a special or customized meaning), and refers to the ability to inhibit precipitation of the active pharmaceutical ingredient either in vitro or in vivo. Preferably, the precipitation inhibitor is different from the dispersing polymer. Examples include hydroxypropyl methyl cellulose succinate, polyvinyl acetate phthalate, Soluplus (polyvinyl caprolactam-polyvinyl acetate-polyethylene glycol graft copolymer (PCL-PVAc-PEG)), Vinylpyrrolidone-vinyl acetate copolymer (Copovidone), polyvinyl pyrrolidone (povidone), cellulose acetate succinate, methyl cellulose acetate succinate, ethyl cellulose acetate succinate, hydroxypropyl cellulose acetate succinate, hydroxypropyl methyl cellulose acetate succinate (HPMCAS), hydroxypropyl cellulose acetate phthalate succinate, cellulose propionate succinate, hydroxypropyl cellulose butyrate succinate, hydroxypropyl methyl cellulose phthalate (HPMCP), cellulose acetate phthalate, methyl cellulose acetate phthalate, ethyl cellulose acetate phthalate, hydroxypropyl cellulose acetate phthalate, hydroxypropyl methyl cellulose acetate phthalate, cellulose propionate phthalate, hydroxypropyl cellulose butyrate phthalate, cellulose acetate trimellitate, methyl cellulose acetate trimellitate, ethyl cellulose acetate trimellitate, hydroxypropyl cellulose acetate trimellitate, hydroxypropyl methyl cellulose acetate trimellitate, hydroxypropyl cellulose acetate trimellitate succinate, cellulose propionate trimellitate, cellulose butyrate trimellitate, cellulose acetate terephthalate, cellulose acetate isophthalate, cellulose acetate pyridinedicarboxylate, salicylic acid cellulose acetate, hydroxypropyl salicylic acid cellulose acetate, ethylbenzoic acid cellulose acetate, hydroxypropyl ethylbenzoic acid cellulose acetate, ethyl phthalic acid cellulose acetate, ethyl nicotinic acid cellulose acetate, ethyl picolinic acid cellulose acetate, carboxy methyl cellulose, carboxy ethyl cellulose, ethyl carboxy methyl cellulose, hydroxypropyl methyl cellulose acetate, hydroxypropyl methyl cellulose (HPMC), hydroxypropyl cellulose, methyl cellulose, hydroxyethyl methyl cellulose, hydroxyethyl cellulose acetate, and hydroxyethyl ethyl cellulose, copolymers of methacrylates, copolymers of acrylates, poly(methacylic acid-co-methyl methacrylate), alone or in combination.

The term “solid form” or “solid” is to be given its ordinary and customary meaning to a person of ordinary skill in the art and not limited to a special or customized meaning), and refers to a composition that is a solid. In particular, the term refers to amorphous dispersions at or below their glass transition temperature. Thus, when at or below their respective glass transition temperatures, SD's and ADGs are solid forms.

The term “wetting agent” is to be given its ordinary and customary meaning to a person of ordinary skill in the art (and not limited to a special or customized meaning), and refers to compounds, which aid in water uptake and thus aid in the disintegration and dissolution process. A “surfactant” may be of any kind as long as it is pharmaceutically applicable, and it may be a mixture of two or more types. A surfactant may improve the solubility of a poorly soluble drug by improving the wettability of the poorly soluble drug. Either an ionic surfactant or a nonionic surfactant may be used. The ionic surfactant is preferably one or more selected from the group consisting of sulfuric ester salt, stearic acid salts, oleic acid salts, benzalkonium chloride, bile acid, carboxylate, sulfates, succinates such as dioctyl sulfosuccinate and sulfonate, more preferably sulfates, and most preferably sodium lauryl sulfate and pluronic F68. A nonionic surfactant is preferably poloxamer, phospholipids, lecithin, glycerol monostearate, Gelucire, Labrasol, Labrafil, Capryol, Lauroglycol, Plurol Oleique. Examples of nonionic surfactants include inulin lauryl carbamate, polyoxyethylene alkyl ethers, polyoxyethylene castor oil derivatives, polyoxyethylene sorbitan fatty acid esters and polyoxyethylene-polyoxypropylene copolymers.

Other suitable excipients may include hydroxypropyl-b-cyclodextrin (HPBCD) and D-a-tocopheryl polyethylene glycol succinate (TPGS).

### EXAMPLES

### Example 1 and Comparative Example A

Preparation of 25/75 (w/w) ADGs of indomethacin and HPMCAS-M 300 g granulation binder of 25/75 (w/w) indomethacin/HPMCAS-M was formed by dissolving crystalline indomethacin (ex MP Biomedicals) at 2 wt % solids in acetone to form a clear, yellow solution while stirring. HPMCAS-M polymer (ex Shin Etsu) at 6 wt % solids was subsequently added and stirred until dissolution. The total solids content of the indomethacin/HPMCAS-M solution was 8 wt %.

Separately, a 40 g bed of a fluidized substrate containing 45 wt % mannitol (Pearlitol 50C ex Roquette), 45 wt % microcrystalline cellulose (Avicel Ph 101 ex FMC), 7.5 wt % croscarmellose sodium (AcDiSol ex FMC) and 2.5 wt % sodium lauryl sulfate (SLS ex Fisher Scientific) was weighed out and charged into the Vector VFC Lab Micro Flo-coater fluidized-bed processor. The fluidized substrate was fluidized using an air flow between 50 and 120 Lpm (temperature 20-30° C.) in the Vector VFC Lab Micro Flo-coater and the granulation binder was atomized via a top spray two-fluid nozzle at a spray rate of 3 g/min (atomizing pressure 14 psi; temperature 20-30° C.) into the fluidized-bed (bed temperature 16-23° C.) of the VFC Lab Micro Flo-coater causing the components to get agglomerated by the drug/polymer granulation binder while evaporating the solvent. The target weight gained by spraying the granulation binder was calculated to be 37.5 wt. % ((binder solids weight)/(binder solids weight+substrate component weight)×100). This resulted in a theoretical potency of the ADGs calculated to be 9.375% indomethacin. The amorphous dispersion granules (ADGs) of Example 1 (in accordance with the present invention) were collected from the product container of the Vector VFC Lab Micro Flo-coater fluidized-bed processor as free-flowing material.

SDIs were obtained using the same indomethacin/HPMCAS-M, i.e. the granulation binder of Example 1, and spray-dried using a Buchi B-290 laboratory scale spray dryer (BUCHI Labortechnik AG), and processed and collected using a cyclone to yield the spray-dried powder of 25/75 (w/w) indomethacin/HPMCAS-M SDIs. The process parameters are summarized in Table 1. The spray-dried powder was transferred to a convection tray dryer and dried at 40° C. for 18 hours to remove residual acetone solvent. The conventional SDIs (Comparative Example A) were collected.

TABLE 1
Representative Spray-drying Process Parameters for
Manufacture of 25% A indomethacin/HPMCAS-M
spray dried intermediate (Comparative Example A)

| | |
|---|---|
| Spray Solvent | Acetone |
| Drying Gas | Nitrogen |
| Inlet Temp. | 80-95° C. |
| Drying Gas Flow Rate | 30-40 m³/hr |
| Outlet Temp. | 43-47° C. |
| Condenser Temp. | −20° C. |
| Spray Solution Concentration | 8 wt. % |
| Spray Rate | ~25 g/min |
| Atomizing Pressure | 20-30 psi |
| Secondary Drying | 12-18 hours at 40° C. |

The ADG of Example 1 and the SDI of Comparative Example A were characterized via modulated differential scanning calorimetry (mDSC) measurements and glass transition (Tg) values were determined to be 126.63° C. and 126.71° C., respectively. In both ADG and SDI no phase separation was observed.

Solid-state characterization via Powder X-Ray Diffraction (PXRD) was further performed. The PXRD data for both Example 1 and Comparative Example A did not show any peaks associated with crystalline indomethacin, indicating that indomethacin was amorphous and homogeneous.

In vitro performance testing of the ADGs and SDIs was measured under non-sink conditions using a gastric-to-intestinal media transfer test. ADGs of Example 1 and Comparative Example A were initially dosed in pH 1.0 in simulated gastric fluid (SGF) at a concentration of 2 mg indomethacin per milliliter and stirred gently. Subsequently, fasted simulated intestinal fluid (FaSSIF, biorelevant.com) media having a pH of 6.8 was added to the vessel resulting in a final concentration of 1 mg indomethacin per milliliter of FaSSIF (at pH 6.8). Sample aliquots were removed from the dissolution vessel at various time points and the free drug and colloidal drug were processed separately. Aliquots were then analyzed using a high-performance liquid chromatography (HPLC) method. Table 2 demonstrates that the indomethacin ADGs of Example 1 show improved solubility in the in-vitro test compared to the pure crystalline indomethacin API and the SDIs of Comparative Example A.

TABLE 2
In-vitro dissolution testing of the
indomethacin ADGs, SDIs and crystalline API

| | Cmax | AUC | Cmax | AUC |
|---|---|---|---|---|
| Material | total | total | free | free |
| Fasted Simulated Gastric | | | | |
| (0.1 N HCl) | | | | |
| Example 1 | 26.6 | 774.7 | 0.0 | 0.0 |
| Comparative Example A | 52.1 | 1359.4 | 0.0 | 0.0 |
| indomethacin crystalline API | 57.7 | 1020.4 | 0.0 | 0.0 |
| Fasted Simulated Intestinal | | | | |
| (FaSSIF, H 6.8) | | | | |
| Example 1 | 1519.9 | 270474.6 | 1387.4 | 239449.6 |
| Comparative Example A | 1434.4 | 255578.3 | 1293.3 | 227153.8 |
| indomethacin crystalline API | 487.9 | 81054.9 | 406.3 | 63324.8 |

Samples of the indomethacin ADGs of Example 1 and SDIs of Comparative Example A were analyzed for particle morphology and size via scanning electron microscopy (SEM) (FEI Quanta 2000). The SEM images of the granules of Example 1 show agglomerates of interconnected substrate particles bound together by the amorphous solid solution of the indomethacin/HPMCAS-M. The SEM images show ADGs of Example 1 with diameters greater than 100 microns. The ADGs so made were free flowing and did not require further processing to manufacture the final tablet dosage form. The SEM images of the spray dried granules of Comparative Example A show particles with sizes ranging from about 1 micron to 25 microns in diameter. These particles are poorly flowing in nature and require further processing as described below for manufacturing a tablet dosage form.

Manufacture of Tablets of ADGs of Example 1 and SDIs of Comparative Example A

Indomethacin ADGs prepared in Example 1 and SDIs prepared in Comparative Example A were used to manufacture tablets utilizing a single punch tablet press (Natoli RD-10). The tablet composition as used for ADGs of Example 1 is shown in Table 3.

TABLE 3
Tablet composition of the ADGs of Example 1

| | Unit Composition | |
|---|---|---|
| Component/Grade | wt. (mg) | % w/w |
| Example 1 | 143.1 | 47.7 |
| Avicel PH 101/NF | 70.8 | 23.6 |
| Pearlitol 50 | 70.8 | 23.6 |
| Ac-Di-Sol/NF | 11.7 | 3.9 |
| Sodium Lauryl Sulfate/NF | 3.9 | 1.3 |
| Total: | 300 | 100.0 |

SDIs prepared in Comparative Example A were used to manufacture tablets utilizing a single punch tablet press (Natoli RD-10). These tablets were manufactured using a typical industry standard multi-step procedure. The procedure was performed using the following multiple unit operations: Add all intragranular (i.g) components, except Mg Stearate, to a container. Manually blend for approximately 1 minute. Delump intragranular (i.g.) blend by passing through a 20 mesh (850-micron) screen. Charge sieved blend back into the container and manually blend for approx. 1 minute. Add Mg Stearate to 20 mesh sieve. Pass the Mg Stearate through the sieve using the delumped i.g. blend. Add blend back into container and manually blend for a minimum of 30 seconds. Dry-granulate the blend by slugging the above blend using a 0.5-in flat-faced (FF) tooling to a tensile strength target of ˜0.4 MPa. Granulate slugs by passing through a 20 mesh sieve to yield the granules. Add extragranular (e.g.) Magnesium Stearate to i.g. granules in a container. Manually blend for minimum of 30 seconds. Compress tablets using 0.4062″ SRC tooling targeting an hardness of ˜10.0-11 kPa (˜1.5 MPa tensile strength). This procedure resulted in a final tablet composition as set forth in Table 4.

TABLE 4
Final tablet composition of SDIs of Comparative Example A

| | Unit Composition | |
|---|---|---|
| Component/Grade | Weight (mg) | % w/w |
| Comparative Example A | 143 | 47.7 |
| Avicel PH 101/NF | 71 | 23.5 |
| Pearlitol 50 | 69 | 23.0 |
| Ac-Di-Sol/NF | 12 | 3.9 |
| Sodium Lauryl Sulfate/NF | 4 | 1.3 |
| Magnesium Stearate/NF | 1 | 0.25 |
| lntragranular Total: | 299 | 99.8 |
| Extra-granular: Magnesium | 1 | 0.25 |
| Stearate/NF | | |
| Total: | 300 | 100.00 |

The tablets manufactured with ADGs of Example 1 and SDIs of Comparative Example A have a nominal potency of about 30 mg indomethacin. The tablets from Example 1 were prepared using a target compression force of 4-5 kN in order to yield tablets of about 10-11 kPa hardness (about 1.5 MPa tensile strength). In comparison, tablets from Comparative Example A were prepared using a force of 10 kN.

These tablets were subsequently tested for disintegration in an USP oscillating apparatus (Varian VK100). The tablet disintegration times of the ADGs of Example 1 in water was ˜150 seconds indicating their immediate release nature.

### Example 2—Preparation of 25/75 (w/w) ADGs of Nifedipine and HPMCAS-M

ADGs of Example 2 were prepared as in Example 1 except that nifedipine is used instead of indomethacin. Also the SDIs of Comparative Example B were prepared similar to the SD's of Comparative Example 1, except that nifedipine is used. The single glass transition temperatures (Tg) for both ADGs were determined using mDSC to be 66.1° C. and 67.2° C. for SDIs of Example 2 and Comparative Example B, respectively. In both samples no phase separation was observed.

Solid-state characterization via Powder X-Ray Diffraction (PXRD) was further performed for the nifedipine ADGs and SDIs. The PXRD data for the ADGs of Example 2 and the SDIs of Comparative Example B did not show any peaks associated with crystalline nifedipine, thus indicating that the ADGs and SDIs were amorphous and homogeneous.

In vitro performance testing of the ADGs and SDIs was measured under non-sink conditions as described above for Example 1 and Comparative Example A. The data is shown in Table 5 demonstrates that the nifedipine ADGs and SDIs show improved solubility in the in-vitro test compared to the pure crystalline nifedipine API.

TABLE 5
In-vitro dissolution testing of the nifedipine
ADGs, SDIs and crystalline API

| | Cmax | AUC | Cmax | AUC |
|---|---|---|---|---|
| Material | total | total | free | free |
| Fasted Simulated Gastric | | | | |
| (0.1 N HCl) | | | | |
| Example 2 | 97.4 | 2900.2 | 28.5 | 738.1 |
| Comparative Example B | 122.4 | 3226.9 | 44.8 | 1068.3 |
| indomethacin crystalline API | 17.1 | 483.4 | 2.9 | 68.9 |
| Fasted Simulated Intestinal | | | | |
| (FaSSIF, pH 6.8) | | | | |
| Example 2 | 293.9 | 18927.0 | 74.2 | 6596.1 |
| Comparative Example B | 287.6 | 21257.6 | 89.2 | 8554.4 |
| indomethacin crystalline API | 31.6 | 2810.0 | 8.9 | 1349.5 |

Samples of the indomethacin ADGs of Example 2 and SDIs of Comparative Example B were analyzed for particle morphology and size via scanning electron microscopy (SEM) (FEI Quanta 2000). The SEM images of the granules of Example 2 show agglomerates of interconnected substrate particles bound together by the amorphous solid solution of the indomethacin/HPMCAS-M. The SEM images show ADGs of Example 2 with diameters greater than 100 microns. The ADGs so made were free flowing and did not require further processing to manufacture the final tablet dosage form. The SEM images of the spray dried granules of Comparative Example B show particles with sizes ranging from about 1 micron to 25 micron in diameter. These SDI particles are poorly flowing in nature and require further processing as described below for manufacturing a tablet dosage form.

Manufacture of Tablets of 25/75 (w/w) Nifedipine/HPMCAS-M ADGs

The nifedipine ADGs of Example 2 and SDIs of Comparative Example B were prepared in a similar manner as their respective ADGs of Example 1 and SDIs of Comparative Example A, except that the amounts used are different as shown in Tables 6 and 7.

TABLE 6
Tablet composition of the ADGs of Example 2

| | Unit Composition | |
|---|---|---|
| Component/Grade | wt. (mg) | % w/w |
| 25% A NIF/HPMCAS-M | 110.1 | 36.7 |
| Avicel PH 101/NF | 85.5 | 28.5 |
| Pearlitol 50 | 85.5 | 28.5 |
| Ac-Di-Sol/NF | 14.1 | 4.7 |
| Sodium Lauryl Sulfate/NF | 4.8 | 1.6 |
| Total: | 300 | 100 |

TABLE 7
Final tablet composition of the
ADGs of Comparative Example B

| | Unit Composition | |
|---|---|---|
| Component/Grade | Weight (mg) | % w/w |
| 25% NIF:HPMCAS-M SDI | 110 | 36.73 |
| Avicel PH 101/NF | 85 | 28.46 |
| Pearlitol 50 | 84 | 27.99 |
| Ac-Di-Sol/NF | 14 | 4.74 |
| Sodium Lauryl Sulfate/NF | 5 | 1.58 |
| Magnesium Stearate/NF | 1 | 0.25 |
| Intragranular Total: | 299 | 99.8 |
| Extra granular Magnesium | 1 | 0.25 |
| Stearate/NF | | |
| Total | 300 | 100 |

Prior to tablet manufacturing, hardness-compression profiles of the ADGs and the dry-granulated (slugged) SDIs were measured.

Tablets of 300+/−5 mg weight and a nominal potency of ˜30 mgA, using the composition shown in Table 5, were manufactured using 0.4062″ standard round concave (SRC) tooling punches on a single punch tablet press (Natoli RD-10). A target compression force of 3 kN was selected in order to yield tablets of ˜10-11 kPa hardness (˜1.5 MPa) tensile strength). These tablets were subsequently tested for disintegration in an USP oscillating apparatus (Varian VK100). The tablet disintegration times in water was rapid ˜84 seconds indicating their immediate release nature.

The tablets were tested for dissolution using a sink dissolution test (USP apparatus 2, 50 mM Sodium Phosphate+2% w/v SLS, Paddles at 75 rpm).

### Example 10—Manufacture of 25/75 (w/w) Nifedipine Tablets from SDIs

SDIs prepared in Comparative Example B used to manufacture tablets utilizing a single punch tablet press (Natoli RD-10). These tablets were manufactured using a typical industry standard multi-step procedure. The procedure was performed using the following multiple unit operations: Add all intragranular (i.g) components, except Mg Stearate, to a container. Manually blend for approximately 1 minute. Delump intragranular (i.g.) blend by passing through a 20 mesh (850-microns) screen. Charge sieved blend back into the container and manually blend for approx. 1 minute. Add Mg Stearate to 20 mesh sieve. Pass the Mg Stearate through the sieve using the delumped i.g. blend. Add blend back into container and manually blend for a minimum of 30 seconds. Dry-granulated the blend by slugging the above blend using a 0.5-in FF tooling to a tensile strength target of ˜0.4 MPa. Granulate slugs by passing through a 20 mesh sieve to yield the granules. Add extragranular (e.g.) Magnesium Stearate to i.g. granules in a container. Manually blend for minimum of 30 seconds. Compress tablets using 0.4062″ SRC tooling targeting an hardness of ˜10.0-11 kPa (˜1.5 MPa tensile strength). The procedure resulted in a final tablet composition as set forth in Table 8.

TABLE 8
Final tablet composition of SDIs
of Comparative Example B

| | Unit Composition | |
|---|---|---|
| Component/Grade | Weight (mg) | % w/w |
| Comparative Example B | 110 | 36.73 |
| Avicel PH 101/NF | 85 | 28.46 |
| Pearlitol 50 | 84 | 27.99 |
| Ac-Di-Sol/NF | 14 | 4.74 |
| Sodium Lauryl Sulfate/NF | 5 | 1.58 |
| Magnesium Stearate/NF | 1 | 0.25 |
| Intragranular Total: | 299 | 99.8 |
| Extra granular Magnesium | 1 | 0.25 |
| Stearate/NF | | |
| Total | 300 | 100 |

Prior to manufacturing, hardness-compression profiles of the dry-granulated SDIs were measured. Based on this data, a target compression force of 10 kN was chosen to manufacture the tablets containing SDI of Comparative Example B.

The tablets containing SDI of Comparative Example B were tested for dissolution using a sink dissolution test (USP apparatus 2, 50 mM Sodium Phosphate+2% w/v SLS, Paddle speed=75 rpm).

The non-sink dissolution performance of ADGs, SDI and pure crystalline nifedipine as described in Example 7 (Table 13A and 13B and Example 2 (Table 17A and Table 17B). The data indicates superior dissolution performance of the ADGs and SDI and enhanced solubility when compared to the poorly soluble crystalline nifedipine.

1.-7. (canceled)

8. A method of preparing pharmaceutically acceptable oral dosage forms comprising amorphous dispersion granules, the method comprising the steps of:
(a) dissolving an active pharmaceutical ingredient and a dispersing polymer in a solvent to form a granulation binder, wherein the granulation binder forms a solid solution of amorphous pharmaceutical ingredient and the polymer;
(b) spraying the granulation binder onto a fluidized substrate comprising at least one first tableting agent comprising a ductile component, a second tableting agent comprising a brittle component and comprising a disintegrant to form amorphous dispersion granules; and
(c) formulating the amorphous dispersion granules into a pharmaceutically acceptable oral dosage form.

9. A method according to claim 8, wherein the pharmaceutically acceptable dosage form is a tablet.

10. The method according to claim 8, wherein the ductile component is microcrystalline cellulose and/or starch.

11. The method according to claim 8, wherein the brittle component is selected from the group consisting of mannitol, lactose, calcium phosphate, Starch 1500, silicas, silicates and mixtures thereof.

12. The method according to claim 8, wherein the disintegrant is selected from the group consisting of croscarmellose sodium, cross-linked carboxymethylcellulose, guar gum, cross-linked polyvinyl pyrrolidone (PVP), hydroxypropyl cellulose, soy polysaccharides, clays, starches, and mixtures thereof.

13. The method according to claim 8, further comprising one or more additional excipient selected from the group consisting of surfactants, glidants, lubricants, wetting agents, pH adjusting agents, antioxidants, precipitation inhibitors, flavoring or food additives, coloring agents, stabilizers, binders, odor controlling agents, and preservatives.

14. The method according to claim 8, wherein the solvent is selected from the group consisting of acetone, methanol, ethanol, isopropyl alcohol, tetrahydrofuran (THF), methylene chloride, water, methyl ethyl ketone, acetonitrile, ethyl acetate, dimethyl sulfoxide, N-methyl-2-pyrrolidone, dimethyl formamide and mixtures thereof.

15. The method according to claim 8, wherein two or more solvents are used to form the granulation binder.

16. The method according to claim 8, wherein the active pharmaceutical ingredient is present in an amount of at least 0.1 weight percent, based on the total weight of the granulation binder.

17. The method according to claim 8, wherein the active pharmaceutical is selected from the group consisting of alprazolam, amiodarone, amlodipine, amprenavir, aprepitant, aripiprazole, astemizole, atenolol, auranofin, azathioprine, azelastine, beclomethasone, belsomra, bexarotene, biperiden, boceprevir, budesonide, buprenorphine, butalbital, calcitriol, carbamezapine, carbidopa, carvedilol, cefotaxime, cephalexin, chlorpromazine, cholestyramine, ciprofloxacin, cisapride, cisplatin, clarithromycin, clofazimine, cefuroxime, clemastine fumarate, clonazepam, clozapine, cyclandelate, cyclosporin, danazol, diazepam, diclofenac, diclofenac sodium, digoxin, dipyridamole, divalproex, dobutamine, doxazosin, doxercalciferol, dronabinol, dutasteride, efavirenz, emalapril, estradiol, etodolac, etoposide, etravirine, everolimus, famotidine, felodipine, fenofibrate, fenoprofen, fentanyl citrate, fexofenadine, finasteride, fluconazole, flunosolide, fluphenazine, flurbiprofen, fluvoxamine, furosemide, glipizide, glyburide, glibendamine, griseofulvin, hydrocortisone, ibuprofen, indomethacin, isosorbide dinitrate, isotretinoin, isradipine, itaconazole, ivacaftor, ketoconazole, ketoprofen, lamotrigine, lansoprazole, ledipasivir, loperamide, lopinavir, loratidine, lorazepam, lovastatin, maropitant, medroxyprogesterone, mefenamic acid, megestrol acetate, mesalamine, methylprednisolone, miconazole, midazolam, mitomycin, mometasone, nabilone, nabumetone, naproxen, nelfinavir mesylate, nicergoline, nifedipine, nimodipine, nintedanib, nitrofurantoin, norfloxacin, nufenamic acid, olaparib, oleanolic acid, omeprazole, paclitaxel, paliperidone, perphenazine, phenytoin, piroxicam, posaconazole, quinapril, quinapril hydrochloride, ramipril, retinal palmitate, risperidone, ritonavir, paracetamol, pericalcitol, praziquantel, saquinavir, sertraline, simeprevir, simvastatin, sirolimus, spironolactone, sulfasolizine, tacrolimus, telaprevir, terbinafine, terfernadine, testosterone, testosterone undecanoate, tipranavir, tolterodine tartrate, tretinoin, triamcinolone, valproic acid, vemurafenib, verapamil, voraconazole, zafirlukast, ziprasidone, and zolpidem.

18. The method according to claim 8, wherein the polymer is selected from the group consisting of hydroxypropyl methyl cellulose succinate, polyvinyl acetate phthalate, Soluplus (polyvinyl caprolactam-polyvinyl acetate-polyethylene glycol graft copolymer (PCL-PVAc-PEG)), Vinylpyrrolidone-vinyl acetate copolymer (Copovidone), polyvinyl pyrrolidone (povidone), cellulose acetate succinate, methyl cellulose acetate succinate, ethyl cellulose acetate succinate, hydroxypropyl cellulose acetate succinate, hydroxypropyl methyl cellulose acetate succinate (HPMCAS), hydroxypropyl cellulose acetate phthalate succinate, cellulose propionate succinate, hydroxypropyl cellulose butyrate succinate, hydroxypropyl methyl cellulose phthalate (HPMCP), cellulose acetate phthalate, methyl cellulose acetate phthalate, ethyl celluloseacetate phthalate, hydroxypropyl cellulose acetate phthalate, hydroxypropyl methyl cellulose acetate phthalate, cellulose propionate phthalate, hydroxypropyl cellulose butyrate phthalate, cellulose acetate trimellitate, methyl cellulose acetate trimellitate, ethyl cellulose acetate trimellitate, hydroxypropyl cellulose acetate trimellitate, hydroxypropyl methyl cellulose acetate trimellitate, hydroxypropyl cellulose acetate trimellitate succinate, cellulose propionate trimellitate, cellulose butyrate trimellitate, cellulose acetate terephthalate, cellulose acetateisophthalate, cellulose acetate pyridinedicarboxylate, salicylic acid cellulose acetate, hydroxypropyl salicylic acid cellulose acetate, ethylbenzoic acid cellulose acetate, hydroxypropylethylbenzoic acid cellulose acetate, ethyl phthalic acid cellulose acetate, ethyl nicotinic acid cellulose acetate, ethyl picolinic acid cellulose acetate, carboxyethyl cellulose, carboxyethyl cellulose, ethyl carboxy methyl cellulose, hydroxypropyl methyl cellulose acetate, hydroxypropylmethyl cellulose (HPMC), hydroxypropyl cellulose, methyl cellulose, hydroxyethyl methyl cellulose, hydroxyethyl cellulose acetate, and hydroxyethyl ethyl cellulose, copolymers of methacrylates, copolymers of acrylates, poly(methacylic acid-co-methyl methacrylate), and mixtures thereof.

19. The method according to claim 8, wherein the polymer is present in an amount of at least 0.1 weight percent (wt %), based on the total weight of the amorphous dispersion granule.

20. The method according to claim 8, wherein the amorphous dispersion granule has a d90 value of at least 1 μm.

21. The method according to claim 8, wherein the weight ratio of active pharmaceutical ingredient to polymer is at least 1:20.

22. The method according to claim 8, wherein the first tableting agent, second tableting agent and disintegrant are present in an amount of at least 0.1 weight percent (wt %), based on the total weight of the amorphous drug dispersion.

23. The method according to claim 8, wherein the granulation binder is sprayed onto fluidized substrate particles in a fluidized bed.

24. The method according to claim 8, wherein the fluidized bed is a circulating fluidized bed, a vibratory fluidized bed or an annular fluidized bed.

25. The method of claim 8, wherein the spraying is through a top spray nozzle, a bottom spray nozzle, or a tangential spray nozzle.

26. The method according to claim 8, wherein the oral dosage forms are selected from the group consisting of capsules, caplets, powders, pellets, and granules.

27. A method of preparing amorphous dispersion granules, the method comprising the steps of:
(a) dissolving an active pharmaceutical ingredient and a dispersing polymer in a solvent to form a granulation binder, wherein the granulation binder forms a solid solution of amorphous pharmaceutical ingredient and the polymer; and
(b) spraying the granulation binder onto a fluidized substrate comprising at least one first tableting agent comprising a ductile component, a second tableting agent comprising a brittle component and comprising a disintegrant to form amorphous dispersion granules.

28. The method according to claim 27, wherein the fluidized substrate comprises one or more additional excipients selected from the group consisting of surfactants, glidants, lubricants, wetting agents, pH adjusting agents, antioxidants, precipitation inhibitors, flavoring or food additives, coloring agents, stabilizers, binders, odor controlling agents, and preservatives.

29. The method according to claim 27, wherein the granulation binder is sprayed onto fluidized substrate particles in a fluidized bed.

30. The method of preparing amorphous dispersion granules according to claim 27, wherein the amorphous dispersion granules have superior compressibility, compactibility and tabletability compared to a spray dried intermediate (SDI) physically mixed with the substrate and dry granulated consisting of the same ingredients in similar amounts to the amorphous dispersion granules.
